(19) United States
(12) Patent Application Publication
(10) Pub. No.: US 2007/0037830 A1
(43) Pub. Date: Feb. 15, 2007

(54) 2-Adamantyl derivatives as p2X7 receptor antagonists.

(75) Inventors: David Cladingboel, Leicestershire (GB); Rhonan Ford, Leicestershire (GB); Paul Willis, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 10/567,711

(22) PCT Filed: Jul. 28, 2004
(86) PCT No.: PCT/SE04/01154

§ 371 Date: Feb. 7, 2006

(30) Foreign Application Priority Data
Aug. 8, 2003 (SE) 0302192-0

(51) Int. Cl. A61K 31/52; A61K 31/505; A61K 31/44; A61K 31/47; A61K 31/4162; A61K 31/381; A61K 31/165; C07D 333/22; C07D 277/62; C07D 215/12

(52) U.S. Cl. 514/263.1; 514/311; 514/419; 514/621; 514/310; 514/367; 514/277; 514/269; 544/277; 544/294; 546/176; 546/285; 549/76; 548/495; 548/152; 548/362.5

(57) ABSTRACT

The invention provides compounds of formula (I) or a pharmaceutically acceptable salt or solvate or pro-drug thereof, wherein A, B, X, n and m have the meanings as defined in the specification. Processes for their preparation; pharmaceutical compositions containing them; and their use in therapy are also described.

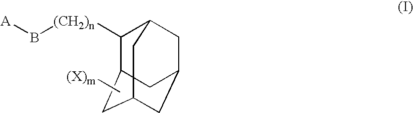

The present invention relates to certain adamantane derivatives, processes for their preparation, pharmaceutical compositions containing them, and their use in therapy. In particular, the present invention relates to 2-adamantyl derivatives effective as P2X₇ receptor antagonists.

The P2X₇ receptor (previously known as P2Z receptor), which is a ligand-gated ion channel, is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, leads to the release of interleukin-1β (IL-1β) and giant cell formation (macrophages/microglial cells), degranulation (mast cells) and proliferation (T cells), apoptosis and L-selectin shedding (lymphocytes). P2X₇ receptors are also located on antigen-presenting cells (APC), keratinocytes, salivary acinar cells (parotid cells), hepatocytes and mesangial cells.

It would be desirable to -make compounds effective as P2X₇ receptor antagonists for use in the treatment of inflammatory, immune or cardiovascular diseases, in the aetiologies of which the P2X₇ receptor may play a role.

1-Adamantyl derivatives having antagonist activity at the P2X₇ receptor are known in the art and are described, for example, in WO 99/29660, WO 00/61569 and WO 01/94338.

The present invention provides a compound of formula (I) 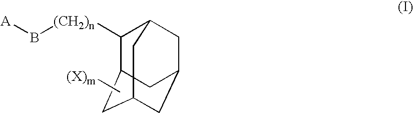
or a pharmaceutically acceptable salt, prodrug or solvate thereof, wherein A represents a phenyl, pyridyl, indolyl, indazolyl, purinyl, pyrimidinyl, thiophenyl, benzothiazolyl, quinolinyl or isoquinolinyl group, each of which may be optionally substituted by one or more substituents, which may be the same or different, selected from halogen, amino, nitro, cyano, hydroxyl, C₁-C₆ alkyl optionally substituted by at least one substituent selected from hydroxyl or halogen, C₁-C₆ alkoxy, or a group of formula
—[Y]ₚ—R¹—R² (II)
where Y represents an oxygen or sulphur atom or a group —N(R³)—;

p is 0 or 1;

R¹ represents a bond or a C₁-C₆ alkyl group which may be optionally substituted by at least one substituent selected from hydroxyl, halogen, C₁-C₆ alkoxy, C₁-C₆ alkylthio, C₁-C₆ hydroxyalkyl, C₁-C₆ hydroxyalkyloxy, C₁-C₆ alkoxycarbonyl, C₃-C₈ cycloalkyl, phenyl (optionally substituted by at least one substituent selected from halogen, hydroxyl and C₁-C₆ alkylsulphonylamino), benzyl, indolyl (optionally substituted by at least one substituent selected from C₁-C₆ alkoxy), oxopyrrolidinyl, phenoxy, benzodioxolyl, phenoxyphenyl, piperidinyl and benzyloxy;

R² represents hydrogen, hydroxyl, —NR⁴R⁵, except that when R¹ represents a bond, then R² represents a saturated or unsaturated 3- to 10-membered ring system which may comprise at least one ring heteroatom selected from nitrogen, oxygen and sulphur, the ring system being optionally substituted by at least one substituent selected from hydroxyl, amino (—NH₂), C₁-C₆ alkyl, C₁-C₆ alkylamino, —NH(CH₂)₂OH, —NH(CH₂)₃OH, NH(CH₂)₄OH, C₁-C₆ hydroxyalkyl, benzyl, and

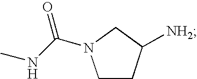

R³ represents a hydrogen atom or a C₁-C₆ alkyl group which may be optionally substituted by at least one substituent selected from hydroxyl, halogen and C₁-C₆ alkoxy;

R⁴ and R⁵ each independently represent hydrogen, pyrrolidinyl, piperidinyl, C₁-C₆ alkylcarbonyl, C₂-C₇ alkenyl, or C₁-C₇ alkyl optionally substituted with at least one substituent selected from carboxyl, hydroxyl, amino (—NH₂), C₁-C₆ alkylamino, di-C₁-C₆ alkylamino, —NH(CH₂)₂OH, C₁-C₆ alkoxy, C₁-C₆ alkylthio, C₁-C₆ alkoxycarbonyl, and a saturated or unsaturated 3- to 10-membered ring system which may comprise at least one ring heteroatom selected from nitrogen, oxygen and sulphur, the ring system being optionally substituted by at least one substituent selected from halogen, hydroxyl, oxo, carboxyl, cyano, C₁-C₆ alkyl, C₁-C₆ hydroxyalkyl, —NR⁶R⁷, —(CH₂)ᵣNR⁸R⁹ and —CONR¹⁰R¹¹,

or R⁴ and R⁵ may together with the nitrogen atom to which they are attached form a saturated 4- to 8-membered heterocyclic ring which may comprise a second ring heteroatom selected from nitrogen and oxygen, the ring being optionally substituted by at least one substituent selected from hydroxyl, halogen, C₁-C₆ alkyl, and C₁-C₆ hydroxyalkyl r is 1, 2, 3, 4, 5 or 6;

R⁶ and R⁷ each independently represent a hydrogen atom or a C₁-C₆ alkyl, C₂-C₆ hydroxyalkyl or C₃-C₈ cycloalkyl group, or R⁶ and R⁷ together with the nitrogen atom to which they are attached form a 3- to 8-membered saturated heterocyclic ring;

R⁸ and R⁹ each independently represent a hydrogen atom or a C₁-C₆ alkyl, C₂-C₆ hydroxyalkyl or C₃-C₈ cycloalkyl group, or R⁸ and R⁹ together with the nitrogen atom to which they are attached form a 3- to 8-membered saturated heterocyclic ring; and

R¹⁰ and R¹¹ each independently represent a hydrogen atom or a C₁-C₆ alkyl, C₂-C₆ hydroxyalkyl or C₃-C₈ cycloalkyl group, or R¹⁰ and R¹¹ together with the nitrogen atom to which they are attached form a 3- to 8-membered saturated heterocyclic ring;

B represents C(O)NH or NHC(O);

n is 1, 2, 3, 4, 5 or 6;

each X is independently selected from halogen or C₁-C₆ alkoxy; and

m is 0, 1, 2, 3, 4, 5, 6, 7, 8, or 9;

with the proviso that when B represents C(O)NH, n is 1 and m is 0, then A is not an unsubstituted phenyl group.

As used herein, the term “halogen” includes fluorine, chlorine, bromine and iodine, and in particular is fluorine or chlorine.

Unless otherwise indicated, the term ‘alkyl’, when used alone or in combination, refers to a straight or branched chain alkyl moiety. A C₁-C₆ alkyl group has from one to six carbon atoms, including methyl, ethyl, n-propyl, isopropyl, butyl, isobutyl, tert-butyl, n-pentyl, n-hexyl and the like. ‘C₁-C₅ alkyl’ and ‘C₁-C₇ alkyl’will be understood accordingly to mean a straight or branched chain alkyl moiety having from one to five or one to seven carbon atoms respectively.

The term ‘cycloalkyl’ refers to a saturated alicyclic moiety having from three to eight carbon atoms and includes, for example, cyclopropyl, cyclobutyl, cyclopentyl and cyclohexyl.

The expression “heterocyclic” includes saturated and unsaturated rings having from 3 up to 10 atoms, at least one of which is a heteroatom selected from oxygen, sulphur or nitrogen. The rings may be mono- or bicyclic and may have alicyclic or aromatic properties. Bicyclic rings may be fully or partially aromatic in character. An unsaturated ring system may be fully or partially unsaturated. Nitrogen heteroatoms will be substituted as necessary, and may also be in the form of N-oxides. Sulphur atoms may be in the form of S, S(O) or S(O₂). Where it is intended that the heterocyclic ring has a maximum number of ring atoms that is less than ten, this is specified. Where it is intended that a ring heteroatom is one of N, S or O in particular, or that the heterocyclic ring comprises one or more ring heteroatoms in specific combination, this is indicated.

‘Optionally substituted’ is used herein to indicate optional substitution by the group or groups specified at any suitable available position.

A “hydroxyalkyl” substituent may contain one or more hydroxyl groups but preferably contains one hydroxyl group.

It will be understood that where B represents C(O)NH the compounds of formula (I) comprise a group A-C(O)NH—(CH₂)ₙ—; and when B represents NHC(O) the compounds of formula (I) comprise a group A-NHC(O)—(CH₂)ₙ—.

In an embodiment of the invention, A represents a phenyl, pyridyl, indolyl or quinolyl group which is optionally substituted as defined above. Preferably, A represents a phenyl, pyridyl or quinolyl group which is optionally substituted as defined above.

Preferred substituents for the ring A include halogen (for example fluorine or chlorine), amino, nitro, cyano, hydroxyl, C₁-C₆ alkyl (for example, methyl, ethyl, propyl, butyl, pentyl or hexyl) optionally substituted by at least one substituent selected from hydroxyl and halogen, C₁-C₆ alkoxy (for example methoxy, ethoxy, propoxy, butoxy, pentoxy or hexoxy), or a group of formula —[Y]ₚ—R¹—R² (II).

Where Y represents —N(R³)—, R³ is a hydrogen atom or a C₁-C₅ alkyl group which may optionally substituted by at least one substituent (for example, one, two or three substituents independently) selected from hydroxyl, halogen (for example, fluorine, chlorine, bromine or iodine) and C₁-C₆ alkoxy (for example, methoxy or ethoxy).

Preferably, R³ represents a hydrogen atom or a C₁-C₅ alkyl group which may be optionally substituted by at least one hydroxyl group.

R¹ represents a bond or a C₁-C₆, preferably C₁-C₄ alkyl group, substituted by at least one substituent (for example one, two or three substituents independently) selected from hydroxyl, halogen (for example, fluorine or chlorine), C₁-C₆ alkoxy(for example, methoxy or ethoxy), C₁-C₆ alkylthio (for example, methyl- or ethyl-thio), C₁-C₆ hydroxyalkyl (such as hydroxymethyl), C₁-C₆ hydroxyalkyloxy, C₁-C₆ alkoxycarbonyl (for example, methoxycarbonyl), C₃-C₈ cycloalkyl (for example, cyclopropyl, cyclobutyl, cyclopentyl or cyclohexyl), phenyl (optionally substituted by at least one substituent selected from halogen, hydroxyl and C₁-C₆ alkylsulphonylamino, such as methylsulphonylamino), benzyl, indolyl (optionally substituted by at least one substituent selected from C₁-C₆ alkoxy such as methoxy), oxopyrrolidinyl, phenoxy, benzodioxolyl, phenoxyphenyl, piperidinyl and benzyloxy.

In an embodiment of the invention, R¹ represents a bond or a C₁-C₄ alkyl group which may be optionally substituted by one, two or three substituents independently selected from hydroxyl, C₁-C₂ alkoxy, methylthio, C₁-C₂ hydroxyalkyl, C₁-C₂ hydroxyalkyloxy, methoxycarbonyl, C₃-C₆ cycloalkyl, phenyl (optionally substituted by at least one substituent selected from halogen, hydroxyl and methylsulphonylamino), benzyl, indolyl (optionally substituted by at least one methoxy), oxopyrrolidinyl, phenoxy, benzodioxolyl, phenoxyphenyl, piperidinyl and benzyloxy.

R² represents hydrogen, hydroxyl, or a group —NR⁴R⁵ except that when R¹ represents a bond, then R² represents a saturated or unsaturated 3- to 10-membered ring system which may comprise at least one ring heteroatom (for example, one, two, three or four ring heteroatoms independently) selected from nitrogen, oxygen and sulphur, the ring system being optionally substituted by at least one substituent (for example, one, two, three or four substituents independently) selected from hydroxyl, amino (—NH₂), C₁-C₆ alkyl, C₁-C₆ alkylamino, —NH(CH₂)₂OH, —NH(CH₂)₃OH, C₁-C₆ hydroxyalkyl, benzyl, and

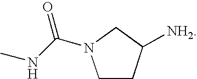

When R² represents a saturated or unsaturated 3 to 10 membered ring system, this ring system is preferably cyclobutyl, cyclohexyl, bicyclo[2.2.1]hept-2-yl, 2,3-dihydro-1H-indenyl, pyrrolidinyl, piperidinyl or piperazinyl.

In an embodiment of the invention, R⁴ and R⁵ each independently represent hydrogen, pyrrolidinyl, C₁-C₂ alkylcarbonyl, C₅-C₇ alkenyl, or C₁-C₇ alkyl optionally substituted with one or two substituents independently selected from carboxyl, hydroxyl, amino, C₁-C₂ alkylamino, di-C₁-C₂ alkylamino, —NH(CH₂)₂OH, C₁-C₂ alkoxy, C₁-C₂ alkylthio, C₁-C₂ alkoxycarbonyl, and a saturated or unsaturated 3- to 10-membered ring system which may comprise at least one ring heteroatom (e.g. one, two, three or four ring heteroatoms independently) selected from nitrogen, oxygen and sulphur, the ring system being optionally substituted by at least one substituent (e.g. one, two, three or four substituents independently) selected from fluorine, hydroxyl, oxo, carboxyl, cyano, C₁-C₂ alkyl, C₁-C₂ hydroxyalkyl, —NR⁶R⁷, —(CH₂)ᵣNR⁸R⁹ and —CONR¹⁰R¹¹.

The saturated or unsaturated 3- to 10-membered ring system referred to in the preceding paragraph is preferably selected from cyclopropyl, cyclohexenyl, phenyl, thienyl, pyridinyl, furyl, bicyclo[2.2.1]hept-5-en-2-yl, 3,4dihydro-2H-pyranyl, thiazolyl, pyrrolyl, pyrazolyl, imidazolyl and thiadiazolyl.

In another embodiment of the invention, where R⁴ and R⁵ together with the nitrogen atom to which they are attached form a saturated six-membered heterocyclic ring, this ring is optionally substituted by at least one substituent (for example, one, two, three or four substituents independently) selected from hydroxyl, halogen (for example, fluorine, chlorine, bromine or iodine), C₁-C₆ alkyl (such as methyl or ethyl), and C₁-C₆ hydroxyalkyl (for example, hydroxymethyl or hydroxyethyl). Preferably, the heterocyclic ring is a piperidinyl, piperazinyl or morpholinyl ring.

R⁶ and R⁷ each independently represent a hydrogen atom, a C₁-C₆ alkyl group such as methyl or ethyl, a C₂-C₆ hydroxyalkyl group (for example, hydroxymethyl) or a C₃-C₈ cycloalkyl group (such as cyclopentyl or cyclohexyl), or R⁶ and R⁷ together with the nitrogen atom to which they are attached form a 3 to 8 membered saturated heterocyclic ring (such as pyrrolidinyl or piperidinyl).

R⁸ and R⁹ each independently represent a hydrogen atom, a C₁-C₆ alkyl group such as methyl or ethyl, a C₂-C₆ hydroxyalkyl group (for example, hydroxymethyl) or a C₃-C₈ cycloalkyl group (such as cyclopentyl or cyclohexyl), or R⁸ and R⁹ together with the nitrogen atom to which they are attached form a 3 to 8 membered saturated heterocyclic ring (such as pyrrolidinyl or piperidinyl).

R¹⁰ and R¹¹ each independently represent hydrogen, a C₁-C₆ alkyl group such as methyl or ethyl, a C₂-C₆ hydroxyalkyl group (for example, hydroxymethyl) or a C₃-C₈ cycloalkyl group (such as cyclopentyl or cyclohexyl), or R₁₀ and R₁₁ together with the nitrogen atom to which they are attached form a 3 to 8 membered saturated heterocyclic ring (such as pyrrolidinyl or piperidinyl).

Preferably, ring A is unsubstituted or substituted by one or more substituents, which may be the same or different, selected from C₁-C₆ alkyl, optionally substituted by at least one substituent selected from halogen or hydroxyl, or C₁-C₆ alkoxy. Where ring A is substituted, it is preferably substituted by one or two substituents, which may be the same or different.

Preferably n is 1 or 2, especially 1.

X is preferably halogen (such as fluorine, chlorine or bromine) or a C₁-C₆ alkoxy group such methoxy or ethoxy. Most preferably, X is halogen or a C₁-C₄ alkoxy group, especially methoxy.

In an embodiment of the invention, m is 1, 2 or 3.

In another embodiment, m is 0.

It will be appreciated that the number and nature of the substituents on rings in the compounds of the invention will be selected so as to avoid sterically undesirable combinations.

In one preferred group of compounds according to the invention, A is phenyl, pyridyl or quinolinyl, optionally substituted by one or more substituents, which may be the same or different, selected from C₁-C₆ alkoxy or C₁-C₆ alkyl, optionally substituted by at least one substituent selected from halogen or hydroxyl; B is NHC(O); m is 0; and n is 1

Preferred compounds of the invention include:—

2-(2-Adamantyl)-N-(5-methoxy-2-methylphenyl)acetamide,

2-(2-Adamantyl)-N-1H-indol-4-ylacetamide,

2-(1-Adamantyl)-N-quinolin-5-ylacetamide,

or a pharmaceutically acceptable salt, prodrug or solvate thereof.

Where the compounds according to the invention contain one or more asymmetrically substituted carbon atoms, the invention includes all stereoisomers, including enantiomers and diastereomers, and mixtures including racemic mixtures thereof. Tautomers and mixtures thereof are also included.

Racemates may be separated into individual enantiomers using known procedures (cf. Advanced Organic Chemistry: 3rd Edition: author J March, p 104-107). A suitable procedure involves formation of diastereomeric derivatives by reaction of the racemic material with a chiral auxiliary, followed by separation, for example by chromatography, of the diastereomers and then cleavage of the auxiliary species.

The compounds according to the invention may be provided as pharmaceutically acceptable salts. Suitable pharmaceutically acceptable salts include base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. In another aspect, where the compound is sufficiently basic, suitable salts include acid addition salts such as methanesulphonate, fumarate, hydrochloride, hydrobromide, citrate, maleate and salts formed with phosphoric and sulphuric acid.

Suitable prodrugs of compounds of formula (I) are compounds which are hydrolysed in vivo to form compounds of formula (I). These may be prepared by conventional methods.

The present invention further provides a process for the preparation of a compound of formula (I) as defined above, or a pharmaceutically acceptable salt, prodrug or solvate thereof, which comprises:

(a) when B represents NHC(O), reacting a compound of formula (III) 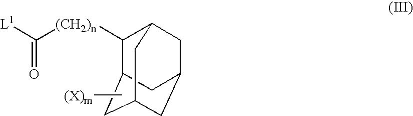
wherein L¹ represents a leaving group (e.g. hydroxyl or halogen) and n,m and X are as defined in formula (I), with a compound of formula (IV), A-NH₂, wherein A is as defined in formula (I); or

(b) when B represents C(O)NH, reacting a compound of formula 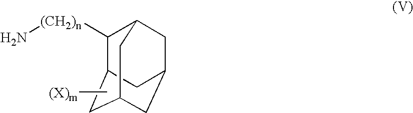
wherein X, m and n are as defined in formula (I), with a compound of formula (VI), A-C(O)-L², wherein L² represents a leaving group (e.g. hydroxyl or halogen) and A is as defined in formula (I); and thereafter, if necessary:

converting the compound obtained into a further compound according to the invention and/or forming a pharmaceutically acceptable salt or prodrug or solvate of the compound.

In processes (a) and (b) the coupling reaction is conveniently carried out in an organic solvent such as dichloromethane, N,N-dimethylformamide or 1-methyl-2-pyrrolidinone. If L¹ or L² represents a hydroxyl group, it may be necessary or desirable to use a coupling agent such as bromo-tris-pyrrolidino-phosphonium hexafluorophosphate (PyBroP).

Compounds of formulae (III) and (V) are known compounds or may be prepared using known techniques by methods analogous to those known in the art. Examples of preparation methods for certain of these compounds are given hereinafter in the examples

Compounds of formula (I) can be converted into further compounds of formula (I) using standard procedures conventional in the art.

It will be appreciated that the preparation of compounds of formula (I) may involve, at various stages, the addition and removal of one or more protecting groups. The protection and deprotection of functional groups is described in ‘Protective Groups in Organic Synthesis’, 2nd edition, T. W. Greene and P. G. M. Wuts, Wiley-Interscience (1991).

The compounds of the invention possess activity as P2X₇ receptor antagonists and are therefore indicated as pharmaceuticals for use in the treatment of rheumatoid arthritis, osteoarthritis, psoriasis, allergic dermatitis, asthma, chronic obstructive pulmonary disease (COPD), hyperresponsiveness of the airway, septic shock, glomerulonephritis, inflammatory bowel disease, Crohn's disease, ulcerative colitis, atherosclerosis, growth and metastases of malignant cells, myoblastic leukaemia, diabetes, Alzheimer's disease, meningitis, osteoporosis, burn injury, ischaemic heart disease, stroke, varicose veins, sarcoidosis, rhinitis, acute and chronic pain, multiple sclerosis, myeloma, bone loss associated with malignancy and inflammatory and neurodegenerative diseases of the eye such as scleritis, episcleritis, uveitis, Sjogrens syndrome-keratoconjuctivitis, sclerokeratitis, optic neuritis, diabetic retinopathy, retinitis pigmentosa, antimalarial—induced retinopathy. They are also advantageous in the treatment of infectious diseases, e.g. anthrax, in particular inflammatory disease caused or exacerbated by bacterial toxins.

According to a further aspect, therefore, the present invention provides a compound of formula (I), or a pharmaceutically acceptable salt, prodrug or solvate thereof, as defined above for use in therapy of the human or animal body.

The invention also provides the use of a compound of formula (I), or a pharmaceutically acceptable salt, prodrug or solvate thereof, as defined above, in the manufacture of a medicament for use in therapy.

It will be appreciated that “therapy” also includes “prophylaxis” unless otherwise indicated. The terms “therapeutic” and “therapeutically” will be understood accordingly

In a further aspect the present invention provides a method of treating a P2X₇ receptor mediated disease condition which comprises administering to a warm-blooded animal a therapeutically effective amount of a compound of the formula (I) or a pharmaceutically acceptable salt, prodrug or solvate thereof.

The invention further provides a method of effecting immunosuppression (e.g. in the treatment of rheumatoid arthritis, osteoarthritis, irritable bowel disease, atherosclerosis or psoriasis) which comprises administering a therapeutically effective amount of a compound of formula (I), or a pharmaceutically acceptable salt or solvate thereof, as hereinbefore defined to a patient.

The invention also provides a method of treating an obstructive airways disease (e.g. asthma or COPD) which comprises administering to a patient a therapeutically effective amount of a compound of formula (I), or a pharmaceutically acceptable salt or solvate thereof, as hereinbefore defined to a patient.

It will be appreciated that dosage administered will vary depending on the compound employed, the mode of administration, the treatment desired and the disorder indicated. Typically, a daily dose of active ingredient in the range of from 0.001 mg/kg to 30 mg/kg body weight is received. This daily dose may be given in divided doses as necessary, the precise amount of the compound received and the route of administration depending on the weight, age and sex of the patient being treated and on the particular disease condition being treated according to principles known in the art.

The compounds of formula (I) and pharmaceutically acceptable salts, prodrug and solvates thereof may be used on their own but will generally be administered in the form of a pharmaceutical composition in association with a pharmaceutically acceptable adjuvant, diluent or carrier. Depending on the mode of administration, the pharmaceutical composition will preferably comprise from 0.05 to 99% w (per cent by weight), more preferably from 0.10 to 70% w, of active ingredient, and, from 1 to 99.95% w, more preferably from 30 to 99.90% w, of a pharmaceutically acceptable adjuvant, diluent or carrier, all percentages by weight being based on total composition.

The present invention therefore also provides a pharmaceutical composition comprising a compound of formula (I), or a pharmaceutically acceptable salt, prodrug or solvate thereof in association with a pharmaceutically acceptable adjuvant, diluent or carrier.

The pharmaceutical compositions of the invention may be administered in standard manner for the disease condition that it is desired to treat, for example by oral, topical, parenteral, buccal, nasal, vaginal or rectal adminstration or by inhalation. For these purposes the compounds of this invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions.

In addition to the compounds of the present invention the pharmaceutical composition of the invention may also contain, or be co-administered (simultaneously or sequentially) with, one or more pharmacological agents of value in treating one or more disease conditions referred to above. Typically unit dosage forms will contain about 1 mg to 500 mg of a compound according to the invention.

Thus, the invention further relates to combination therapies for the treatment of any one of rheumatoid arthritis, osteoarthritis, osteoporosis, psoriasis, inflammatory bowel diseases, COPD, asthma, allergic rhinitis or cancer or the neurodegenerative diseases such as multiple sclerosis, Alzheimer's disease or stroke.

For the treatment of rheumatoid arthritis, the compounds of the invention may suitably be combined with “biological agents” such as TNF-α inhibitors such as anti-TNF monoclonal antibodies (such as Remicade, CDP-870 and Humira) and TNF receptor immunoglobulin molecules (such as Enbrel.reg.). IL-1 receptor antagonist (such as Anakinra) and IL-1 trap, IL-18 receptor, anti-IL-6 Ab, anti-CD20 Ab, anti-IL-15 Ab and CTLA4Ig.

Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin. The COX-2 inhibitors (such as meloxicam, celecoxib, rofecoxib, valdecoxib and etoricoxib) and the cylcooxygenase inhibiting nitric oxide donors (CINOD's) and the “disease modifying agents” (DMARDs) such as methotrexate, sulphasalazine, cyclosporine A, lefunomide; ciclesonide; hydroxychloroquine, d-penicillamine, auranofin or parenteral or oral gold.

The present invention also relates to the combination of a compound of the invention together with a leukotriene biosynthesis inhibitor, 5-lipoxygenase (5-LO) inhibitor or 5-lipoxygenase activating protein (FLAP) antagonist selected from the group consisting of zileuton; ABT-761; fenleuton; tepoxalin; Abbott-79175; Abbott-85761; N-(5-substituted)-thiophene-2-alkylsulfonamides; 2,6-di-tert-butylphenol hydrazones; methoxytetrahydropyrans such as Zeneca ZD-2138; the compound SB-210661; pyridinyl-substituted 2n cyanonaphthalene compounds such as L-739,010; 2-cyanoquinoline compounds such as L-746,530; indole and quinoline compounds such as MK-591, MK-886, and BAY x 1005.

The present invention still further relates to the combination of a compound of the invention together with a receptor antagonists for leukotrienes LTB₄, LTC₄, LTD₄, and LTE₄ selected from the group consisting of the phenothiazin-3-ones such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention together with a PDE4 inhibitor including inhibitors of the isoform PDE4D.

The present invention also relates to the combination of a compound of the invention together with a antihistaminic H₁ receptor antagonists including cetirizine, loratadine, desloratadine, fexofenadine, astemizole, azelastine, and chlorpheniramine.

The present invention still further relates to the combination of a compound of the invention together with a gastroprotective H₂ receptor antagonist or the proton pump inhibitors (such as omeprazole)

The present invention still further relates to the combination of a compound of the invention together with an α₁- and α₂-adrenoceptor agonist vasoconstrictor sympathomimetic agent, including propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of the invention together with anticholinergic agents including ipratropium bromide; tiotropium bromide; oxitropium bromide; pirenzepine; and telenzepine.

The present invention still further relates to the combination of a compound of the invention together with a β₁- to β₄-adrenoceptor agonists including metaproterenol isoproterenol, isoprenaline, albuterol, salbutamol, formoterol, salmeterol, terbutaline, orciprenaline, bitolterol mesylate, and pirbuterol; or methylxanthanines including theophylline and aminophylline; sodium cromoglycate; or muscarinic receptor (M1, M2, and M3) antagonist.

The present invention still further relates to the combination of a compound of the invention together with other modulators of chemokine receptor function such as CCR1, CCR2, CCR2A, CCR2B, CCR3, CCR4, CCR5, CCR6, CCR7, CCR8, CCR9, CCR10 and CCR11 (for the C—C family); CXCR1, CXCR3, CXCR4 and CXCR5 (for the C—X—C family) and CX₃CR1 for the C—X₃—C family.

The present invention still further relates to the combination of a compound of the invention together with an insulin-like growth factor type I (IGF-1) mimetic.

The present invention still further relates to the combination of compound of the invention together with an inhaled glucocorticoid with reduced systemic side effects, including prednisone, prednisolone, flunisolide, triamcinolone acetonide, beclomethasone dipropionate, budesonide, fluticasone propionate, and mometasone furoate.

The present invention still further relates to the combination of a compound of the invention together with (a) tryptase inhibitors; (b) platelet activating factor (PAF) antagonists; (c) interleukin converting enzyme (ICE) inhibitors; (d) IMPDH inhibitors; (e) adhesion molecule inhibitors including VLA-4 antagonists; (f) cathepsins; (g) MAP kinase inhibitors; (h) glucose-6 phosphate dehydrogenase inhibitors; (i) kinin-B₁- and B₂-receptor antagonists; (j) anti-gout agents, e.g., colchicine; (k) xanthine oxidase inhibitors, e.g., allopurinol; (I) uricosuric agents, e.g., probenecid, sulfinpyrazone, and benzbromarone; (m) growth hormone secretagogues; (n) transforming growth factor (TGFβ); (o) platelet-derived growth factor (PDGF); (p) fibroblast growth factor, e.g., basic fibroblast growth factor (bFGF); (q) granulocyte macrophage colony stimulating factor (GM-CSF); (r) capsaicin cream; (s) Tachykinin NK₁ and NK₃ receptor antagonists selected from the group consisting of NKP-608C; SB-233412 (talnetant); and D-4418; and (t) elastase inhibitors selected from the group consisting of UT-77 and ZD-0892 (u) induced nitric oxide synthase inhibitors (iNOS) or (v) chemoattractant receptor-homologous molecule expressed on TH2 cells, (CRTH2 antagonists).

The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11).

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, induced nitric oxide synthase inhibitors (iNOS inhibitors), COX-2 inhibitors such as celecoxib, valdecoxib, rofecoxib and etoricoxib, and the cylco-oxygenase inhibiting nitric oxide donors (CINOD's) analgesics (such as paracetamol and tramadol), cartilage sparing agents such as diacerein, doxycyline and glucosamine, and intra-articular therapies such as corticosteroids and hyaluronic acids such as hyalgan and synvisc.

The compounds of the invention can also be used in combination with existing therapeutic agents for the treatment of inflammatory bowel diseases (Ulcerative colitis and Crohn's disease). Suitable agents to be used include sulphasalazine, 5-amino-salicylates, the thiopurines, azathioprine and 6-mecaptorurine and corticosteroids such as budesonide.

The compounds of the present invention may also be used in combination with anticancer agents such as endostatin and angiostatin or cytotoxic drugs such as adriamycin, daunomycin, cis-platinum, etoposide, taxol, taxotere and farnesyl transferase inhibitors, VegF inhibitors, COX-2 inhibitors and antimetabolites such as methotrexate antineoplastic agents, especially antimitotic drugs including the vinca alkaloids such as vinblastine and vincristine;

The compounds of the invention may also be used in combination with antiviral agents such as Viracept, AZT, aciclovir and famciclovir, and antisepsis compounds such as Valant.

The compounds of the present invention may also be used in combination with cardiovascular agents such as calcium channel blockers, lipid lowering agents such as statins, fibrates, beta-blockers, Ace inhibitors, Angiotensin-2 receptor antagonists and platelet aggregation inhibitors.

The compounds of the present invention may also be used in combination with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti Alzheimer's drugs such as donepezil, tacrine, COX-2 inhibitors, propentofylline or metryfonate.

The compounds of the present invention may also be used in combination with osteoporosis agents such as roloxifene, droloxifene, lasofoxifene or fosomax and immunosuppressant agents such as FK-506, rapamycin, cyclosporine, azathioprine, and methotrexate;.

The activity and selectivity of the compounds according to the invention may be determined using an appropriate assay as described, for example in WO 99/29660.

The invention is further illustrated by the following non-limiting examples.

The relevant starting materials are commercially available or may be made by any convenient method as described in the literature or known to the skilled chemist or described in the Examples herein.

### EXAMPLE 1

### 2-(2-Adamantyl)-N-(5-methoxy-2-methylphenyl)acetamide

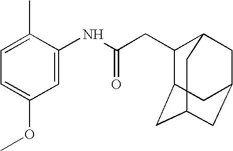

Thionyl chloride (3.0 mL) and 2-adamantylacetic acid (96 mg) were heated together at 79° C. for 5 minutes with stirring before cooled to room temperature and concentrated. The residue was redissolved in dichloromethane (5 mL) and added to a solution of 5-methoxy-2-methylaniline (180 mg) and triethylamine (2.0 mL) in dichloromethane (10 mL). After stirring for 1 hour, dichloromethane (60 mL) and aqueous hydrochloric acid (40 mL, 2 M) were added, the layers were separated and the organic fraction was dried, filtered and evaporated. Purification (SiO₂, dichloromethane) gave the titile compound (0.12 g)

¹H NMR (400 MHz, d₆DMSO) δ 9.15 (1H, s), 7.08 (1H, d), 7.05 (1H, d) 6.65 (1H, dd), 3.69 (3H, s), 2.49 (2H, m), 2.21 (1H, m), 2.10 (3H, s), 2.00-1.92 (2H, m), 1.89-1.67 (10H, m), 1.56-1.49 (2H, m).

MS: APCI (+ve) 314 (M+H⁺)

MP: 193-196° C.

### EXAMPLE 2

### 2-(2-Adamantyl)-N-1H-indol-4-ylacetamide

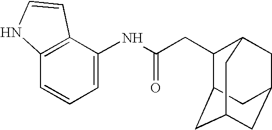

To a solution of 1H-indol-4-amine (20 mg) in dimethylformamide (5 mL) were added 1-(3-dimethylaminopropyl)-3-ethylcarbodiimide hydrochloride (29 mg), N,N-dimethylpyridin-4-amine (18.5 mg) and 2-adamantylacetic acid (29 mg) with continuous stirring. The mixture was heated to 60° C. for 2 hours under nitrogen. The reaction was subsequently cooled to room temperature and then partitioned between ethyl acetate (20 mL) and water (20 mL). The aqueous phase was further extracted with ethyl acetate (2×20 mL) and the combined organics were washed with aqueous potassium hydrogen sulphate (2×10 mL, 2 M), saturated aqueous sodium bicarbonate (2×10 mL) and brine (20 mL). The organics were then dried, filtered and evaporated under vacuum to give the title compound as a white solid (23 mg).

¹H NMR (300 MHz, CDCl₃) δ 7.77-7.75 (1H, m), 7.22-7.14 (3H,m), 6.47 (1H, d), 2.60 (2H, d), 2.40 (1H, bt), 1.96-1.86 (4H,m), 1.76 (2H, s), 1.53 (6H,s).

MS: APCI (+ve) 309 (M+H⁺).

MP: 206-208° C.

### EXAMPLE 3

### 2-(1-Adamantyl)-N-quinolin-5-ylacetamide

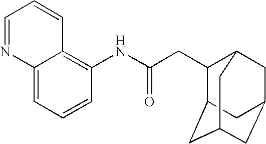

Prepared according to the method of example 2 using 5-quinolinamine.

MS: APCI (+ve) 321 (M+H⁺).

Pharmacological Analysis

Certain compounds such as benzoylbenzoyl adenosine triphosphate (bbATP) are known to be agonists of the P2X₇ receptor, effecting the formation of pores in the plasma membrane (Drug Development Research (1996), 37(3), p. 126). Consequently, when the receptor is activated using bbATP in the presence of ethidium bromide (a fluorescent DNA probe), an increase in the fluorescence of intracellular DNA-bound ethidium bromide is observed. The increase in fluorescence can be used as a measure of P2X₇ receptor activation and therefore to quantify the effect of a compound on the P2X₇ receptor.

In this manner, the title compounds of the Examples were tested for antagonist activity at the P2X₇ receptor. The test was performed in 96-well flat bottomed microtitre plates, the wells being filled with 250 μl of test solution comprising 200 μl of a suspension of THP-1 cells (2.5×10⁶ cells/ml) containing 10⁻⁴M ethidium bromide, 25 μl of a high potassium buffer solution containing 10⁻⁵M bbATP, and 25 μl of the high potassium buffer solution containing 3×10⁻⁵M test compound. The plate was covered with a plastics sheet and incubated at 37° C. for one hour. The plate was then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) were used separately in the test as controls. From the readings obtained, a pIC₅₀ figure was calculated for each test compound, this figure being the negative logarithm of the concentration of test compound necessary to reduce the bbATP agonist activity by 50%. Each of the compounds of the Examples demonstrated antagonist activity, having a pIC₅₀ figure greater than 6.

1. A compound of formula 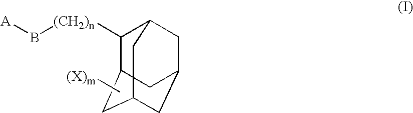
or a pharmaceutically acceptable salt, prodrug or solvate thereof, wherein
A represents a phenyl, pyridyl, indolyl, indazolyl, purinyl, pyrimidinyl, thiophenyl, benzothiazolyl, quinolinyl or isoquinolinyl group, each of which may be optionally substituted by one or more substituents, which may be the same or different, selected from halogen, amino, nitro, cyano, hydroxyl, C₁-C₆ alkyl optionally substituted by at least one substituent selected from hydroxyl or halogen, C₁-C₆ alkoxy, or a group of formula
—[Y]ₚ—R¹—R² (II)
where Y represents an oxygen or sulphur atom or a group —N(R³)—;
p is 0 or 1;
R¹ represents a bond or a C₁-C₆ alkyl group which may be optionally substituted by at least one substituent selected from hydroxyl, halogen, C₁-C₆ alkoxy, C₁-C₆ alkylthio, C₁-C₆ hydroxyalkyl, C₁-C₆ hydroxyalkyloxy, C₁-C₆ alkoxycarbonyl, C₃-C₈ cycloalkyl, phenyl (optionally substituted by at least one substituent selected from halogen, hydroxyl and C₁-C₆ alkylsulphonylamino), benzyl, indolyl (optionally substituted by at least one substituent selected from C₁-C₆ alkoxy), oxopyrrolidinyl, phenoxy, benzodioxolyl, phenoxyphenyl, piperidinyl and benzyloxy;
R² represents hydrogen, hydroxyl, or a group —NR⁴R⁵ except that when R¹ represents a bond, then R² represents a saturated or unsaturated 3- to 10-membered ring system which may comprise at least one ring heteroatom selected from nitrogen, oxygen and sulphur, the ring system being optionally substituted by at least one substituent selected from hydroxyl, amino (—NH₂), C₁-C₆ alkyl, C₁-C₆ alkylamino, —NH(CH₂)₂OH, —NH(CH₂)₃OH, NH(CH₂)₄OH, C₁-C₆ hydroxyalkyl, benzyl, and 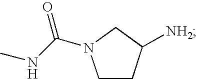
R³ represents a hydrogen atom or a C₁-C₆ alkyl group which may be optionally substituted by at least one substituent selected from hydroxyl, halogen and C₁-C₆ alkoxy;
R⁴ and R⁵ each independently represent hydrogen, pyrrolidinyl, piperidinyl, C₁-C₆ alkylcarbonyl, C₂-C₇ alkenyl, or C₁-C₇ alkyl optionally substituted with at least one substituent selected from carboxyl, hydroxyl, amino (—NH₂), C₁-C₆ alkylamino, di-C₁-C₆ alkylamino, —NH(CH₂)₂OH, C₁-C₆ alkoxy, C₁-C₆ alkylthio, C₁-C₆ alkoxycarbonyl, and a saturated or unsaturated 3- to 10-membered ring system which may comprise at least one ring heteroatom selected from nitrogen, oxygen and sulphur, the ring system being optionally substituted by at least one substituent selected from halogen, hydroxyl, oxo, carboxyl, cyano, C₁-C₆ alkyl, C₁-C₆ hydroxyalkyl, —NR⁶R⁷, —(CH₂)ᵣNR⁸R⁹ and —CONR¹⁰R¹¹,
or R⁴ and R⁵ may together with the nitrogen atom to which they are attached form a saturated 4- to 8-membered heterocyclic ring which may comprise a second ring heteroatom selected from nitrogen and oxygen, the ring being optionally substituted by at least one substituent selected from hydroxyl, halogen, C₁-C₆ alkyl, and C₁-C₆ hydroxyalkyl;
r is 1, 2, 3, 4, 5 or 6;
R⁶ and R⁷ each independently represent a hydrogen atom or a C₁-C₆ alkyl, C₂-C₆ hydroxyalkyl or C₃-C₈ cycloalkyl group, or R⁶ and R⁷ together with the nitrogen atom to which they are attached form a 3- to 8-membered saturated heterocyclic ring;
R⁸ and R⁹ each independently represent a hydrogen atom or a C₁-C₆ alkyl, C₂-C₆ hydroxyalkyl or C₃-C₈ cycloalkyl group, or R⁸ and R⁹ together with the nitrogen atom to which they are attached form a 3- to 8-membered saturated heterocyclic ring; and
R¹⁰ and R¹¹ each independently represent a hydrogen atom or a C₁-C₆ alkyl, C₂-C₆ hydroxyalkyl or C₃-C₈ cycloalkyl group, or R¹⁰ and R¹¹ together with the nitrogen atom to which they are attached form a 3- to 8-membered saturated heterocyclic ring;
B represents C(O)NH or NHC(O);
n is 1, 2, 3, 4, 5 or 6;
each X is independently selected from halogen or C₁-C₆ alkoxy; and
m is 0, 1, 2, 3, 4, 5, 6, 7, 8, or 9;
with the proviso that when B represents C(O)NH, n is 1 and m is 0, then A is not an unsubstituted phenyl group.

2. A compound according to claim 1 wherein A represents a substituted or unsubstituted group selected from phenyl, pyridyl, indolyl or quinolinyl group.

3. A compound according to claim 1 wherein A is substituted by one or more substituents, which may be the same or different, selected from C₁-C₆ alkoxy or C₁-C₆ alkyl, optionally substituted by at least one substituent selected from halogen or hydroxyl.

4. A compound according to claim 1 wherein B represents NHC(O).

5. A compound according to claim 1 wherein m is 1, 2 or 3.

6. A compound according to claim 5 wherein X is halogen or C₁-C₄ alkoxy.

7. A compound according to claim 1 wherein m is 0.

8. A compound according to claim 1 wherein n is 1 or 2.

9. A compound of formula (I) according to claim 1 which is selected from the group consisting of
2-(2-Adamantyl)-N-(1H-indol-4-yl)acetamide, and
2-(2-Adamantyl)-N-(5-methoxy-2-methylphenyl)acetamide, and
2-(1-Adamantyl)-N-quinolin-5-ylacetamide,
or a pharmaceutically acceptable salt, prodrug or solvate thereof.

10. A pharmaceutical composition comprising a compound of formula (I), or a pharmaceutically acceptable salt, prodrug or solvate thereof, as claimed in claim 1, in association with a pharmaceutically acceptable adjuvant, diluent or carrier.

11. A pharmaceutical composition comprising a compound of formula (I) or a pharmaceutially acceptable salt, pro-drug or solvate thereof, as claimed in claim 1, in combination with one or more additional pharmaceutically active agents.

12. A process for the preparation of a pharmaceutical composition as claimed in claim 10 which comprises mixing a compound of formula (I), or a pharmaceutically acceptable salt, prodrug or solvate thereof, as defined claim 1 with a pharmaceutically acceptable adjuvant, diluent or carrier.

13. (canceled)

14. A method of treating a disease condition mediated by the P2X₇ receptor, the method comprising administering a therapeutically effective amount of a compound of formula (I), or a pharmaceutically acceptable salt, prodrug or solvate thereof, as claimed in claim 1.

15-16. (canceled)

17. A method of treating osteoarthritis, the method comprising administering a therapeutically effective amount of a compound of formula (I) or a pharmaceutically acceptable salt, prodrug or solvate thereof as claimed in claim 1.

18. A method of treating rheumatoid arthritis, the method comprising administering a therapeutically effective amount of a compound of formula (I), or a pharmaceutically acceptable salt, prodrug or solvate thereof, as claimed in claim 1.

19. A method of treating artherosclerosis, the method comprising administering a therapeutically effective amount of a compound of formula (I) or a pharmaceutically acceptable salt, prodrug or solvate thereof as claimed in claim 1.

20. A method of treating rheumatoid arthritis or osteoarthritis which comprises administering to a patient a therapeutically effective amount of a compound of formula (I) or a pharmaceutically acceptable salt, prodrug or solvate thereof as claimed in claim 1.

21. A method of treating an obstructive airways disease which comprises administering to a patient a therapeutically effective amount of a compound of formula (I) or a pharmaceutically acceptable salt, prodrug or solvate thereof as claimed in claim 1.

22. A process for the preparation of a compound of formula (I) as claimed in claim 1 or a pharmaceutically acceptable salt, prodrug or solvate thereof, which comprises:
(a) when B represents NHC(O), reacting a compound of formula (III) 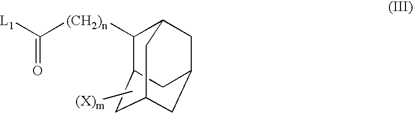
wherein L¹ represents a leaving group and n, m and X are as defined in formula (I), with a compound of formula (IV), A-NH₂, wherein A is as defined in formula (I); or
(b) when B represents C(O)NH, reacting a compound of formula 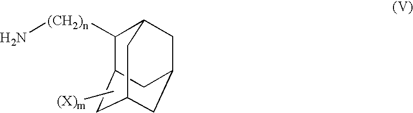
wherein X, m and n are as defined in formula (I), with a compound of formula (VI), A-C(O)-L², wherein L² represents a leaving group and A is as defined in formula (I); and optionally thereafter carrying out one or more of the following:
converting the compound obtained into a further compound according to the invention and/or
forming a pharmaceutically acceptable salt or prodrug or solvate of the compound.

23. The method of claim 21, wherein the obstructive airways disease is asthma or chronic obstructive pulmonary disease.
